(19) United States
(12) Patent Application Publication
(10) Pub. No.: US 2026/0114821 A1
(43) Pub. Date: Apr. 30, 2026

(54) Method for determining the physiological states of an operator, and associated computer program and determination system

(71) Applicant: THALES, Meudon (FR)

(72) Inventors: Victor NIAUSSAT, MERIGNAC (FR); Bastien BERTHELOT, MERIGNAC (FR)

(21) Appl. No.: 19/369,956

(22) Filed: Oct. 27, 2025

(30) Foreign Application Priority Data
Oct. 28, 2024 (FR) FR2411755

(51) Int. Cl. A61B 5/00

(52) U.S. Cl. CPC A61B 5/7282

(57) ABSTRACT

A method for determining the physiological state of an operator, a first preliminary step of determining a correspondence matrix between a mathematical model allowing the determination of a physiological state of the operator and a set of data types necessary for implementing the mathematical model. The method further comprises the following steps: verification of the availability of each sensor; when a sensor is unavailable, exclusion of consideration of each mathematical model using the data type corresponding to this sensor, in accordance with the correspondence matrix; and determination of the physiological states of the operator from the mathematical models not excluded from consideration.

### CROSS REFERENCE TO RELATED APPLICATIONS

Priority is claimed from French Patent Application FR2411755 filed on October 28, 2024, the entire disclosure of which is hereby incorporated by reference herein in its entirety.

### FIELD OF THE INVENTION

The present invention relates to a method for determining the physiological states of an operator.

This invention also relates to an associated computer program and a determination system related to this method for determining.

The technical field of the invention is the monitoring of operators evolving in a critical operational context.

### BACKGROUND OF THE INVENTION

The invention can thus be used in any high-sensitivity field in which monitoring the physiological states of an operator presents an interest. This is notably the case in the aeronautical, aerospace, railway, nuclear, medical fields, etc.

As is known, monitoring the physiological states of an operator is a means of analyzing and evaluating their cognitive behavior under operational conditions, or even outside these conditions, notably for longitudinal monitoring.

Generally, such an analysis relies on means allowing the capture and analysis of physiological and cognitive parameters, complemented by context parameters.

Physiological data can present any parameter related to the vitality of the human body such as body temperature, heart rate, oxygen saturation, or even respiratory rate.

These data are generally obtained from a plurality of specific sensors optimized to measure them. Furthermore, these data are generally associated with signal processing treatments such as filtering, allowing to reduce or eliminate various noises in the signals that could degrade the markers to be observed. The aim is to provide the right level of signal processing that will allow to obtain a sufficiently clean signal to be used in models and achieve the desired level of detection or prediction performance.

According to state-of-the-art methods, the problem of monitoring the physiological data of an operator is solved using traditional means such as specific sensors, detection or prediction models, and signal processing algorithms derived from the sensors.

These solutions mainly rely on extracting interesting features from physiological signals, followed by, the use of, detection or prediction algorithms, such as artificial intelligence algorithms, to better separate the detection or prediction of different physiological states.

According to the state of the art, initially, specific sensors are used to capture physiological signals such as heart rate, oxygen saturation, body temperature, etc. Then, signal processing techniques are applied to these signals to reduce unwanted noise and disturbances to obtain higher quality signals.

When these signals are preprocessed, the state of the art proposes extracting relevant features from these physiological signals. These features can include parameters such as heart rate variability, heart rate peaks, oxygen saturation variation, etc. The objective here is to select the most discriminating features that allow to effectively differentiate the different physiological states.

Then, these features are used as input for detection or prediction algorithms, such as artificial intelligence algorithms, which are trained to recognize the characteristic patterns and motifs of each physiological state. These algorithms can include classification and regression techniques, or even neural networks to perform the detection or prediction of specific physiological states.

However, state-of-the-art solutions present a certain number of drawbacks.

Firstly, traditional solutions are often limited by the availability and reliability of sensors.

In particular, in the event of a sensor failure, the analysis of physiological signals can be compromised, which can lead to detection or prediction errors or an inability to detect abnormal physiological states.

Furthermore, existing solutions do not optimally take into account management of sensor availability. In the event of a sensor loss, it is often necessary to stop or completely recalibrate the system, which leads to interruptions in monitoring and delays in restoring normal operation.

### SUMMARY OF THE INVENTION

Existing solutions therefore lack flexibility and the ability to manage cases where detection or prediction models of physiological states are unavailable due to failing sensors.

This means that in the absence of certain sensors, it is difficult, if not impossible, to obtain reliable results on the physiological states of the operator.

The present invention has as its object to address these drawbacks and to propose a solution allowing to determine the physiological states of the operator reliably even when one or more sensors are no longer available.

Furthermore, the solution is particularly flexible in that it can easily adapt to the potential loss of one or more sensors.

To this end, the invention relates to a method for determining the physiological states of the operator;

the method comprising a first preliminary step of determining a correspondence matrix between a mathematical model allowing to determine a physiological state of the operator and a set of data types necessary for implementing the mathematical model;

the method further comprises the following steps:

verification of the availability of each sensor providing a data type;

when a sensor is unavailable, exclusion from consideration of each mathematical model using the data type corresponding to this sensor, in accordance with the correspondence matrix;

determination of the physiological states of the operator from the mathematical models not excluded from consideration.

The physiological states are advantageously all different.

According to other advantageous characteristics of the invention, the method comprises one or more of the following characteristics, taken in isolation or in any technically possible combination:

the method further comprises a step of substituting at least one mathematical model excluded from consideration with a mathematical sub-model allowing to determine the same physiological state from a set of data types without the data type of the unavailable corresponding sensor;

the method further comprises a second preliminary step of determining an incidence matrix indicating the level of incidence of each physiological state on each other physiological state;

each level of incidence corresponds to the probability of transitioning from one physiological state to another physiological state;

the method further comprises a step of determining at least one physiological state corresponding to a mathematical model excluded from consideration, by an aggregation model using the or each physiological state inducing this physiological state corresponding to the mathematical model excluded from consideration, in accordance with the incidence matrix;

said physiological state, corresponding to the mathematical model excluded from consideration, is determined with a confidence level determined as a function of the level of incidence of the or each physiological state used by the aggregation model;

each physiological state is chosen from the group comprising at least:

hypoxia;

loss of consciousness;

stress;

fatigue;

spatial disorientation;

blackout

dehydration;

mental load;

mind wandering;

hyperventilation;

hypoglycemia;

visual or attentional tunneling;

each data type is chosen from the group comprising at least:

Heart rate, notably at different frequencies;

Oxygen saturation;

Electroencephalogram;

Respiratory rate;

Body temperature;

Images;

Acoustic data;

Eye activity;

Head position and acceleration;

Blood glucose levels;

Skin conductance;

Muscle activity;

Photoplethysmography;

Functional near-infrared spectroscopy.

The invention also relates to a computer program including software instructions which, when executed by a computer, implement a method as defined above.

The invention finally relates to a system for determining a physiological state of the operator comprising technical means configured to implement a method as defined above.

### BRIEF DESCRIPTION OF THE DRAWINGS

The invention will become clearer upon reading the following description, given solely by way of non-limiting example, and made with reference to the appended drawings in which:

FIG. 1 is a schematic view of a determination system according to the invention;

FIG. 2 is a flowchart of a method for determining a physiological state of the operator , the method for determining being implemented by the determination system of FIG. 1;

FIGS. 3 to 9 are different illustrations of the implementation of different steps of the method of FIG. 2.

### DETAILED DESCRIPTION OF THE INVENTION

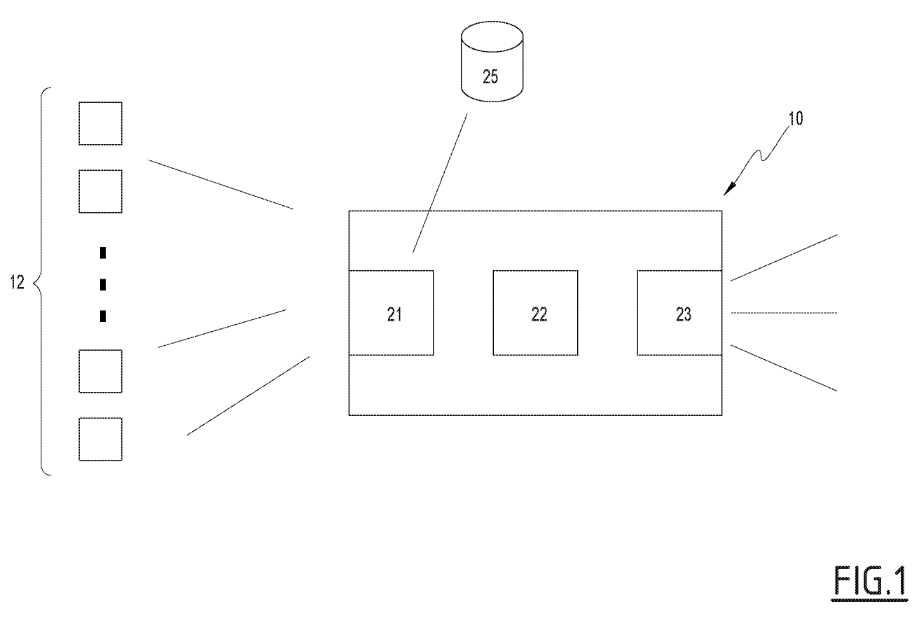

Indeed, FIG. 1 represents a system for determining a physiological state of the operator according to the invention.

By operator, is meant, any person operating a machine, vehicle, or any other sensitive system. Thus, for example, such an operator pilots an aircraft, such as an airplane. In such a case, this operator is an airplane pilot.

Alternatively, the operator is any other crew member or a ground operator performing maintenance on an aircraft.

By aircraft, we mean any flying device that can be piloted from its cockpit, as is the case for example with an airplane or a helicopter, or remotely from it, as is the case for example with a drone.

In general, the notion of the operator can apply to any other person performing a critical mission, for example in the field of transport (railway or heavy goods or any transport for example) or in the nuclear or space field, or in medicine.

The determination system 10 according to the invention allows to determine a plurality of different physiological states of this operator.

In particular, by physiological state, is meant any state of the operator likely to influence their physical or cognitive work.

Each physiological state is, for example, chosen from the group comprising at least:

hypoxia;

loss of consciousness;

stress;

fatigue;

spatial disorientation;

blackout (or G-LOC);

dehydration;

mental load;

mind wandering;

hyperventilation;

hypoglycemia;

visual or attentional tunneling.

The determination system 10 allows to determine a physiological state of the operator from a plurality of data provided by a plurality of sensors 12.

These sensors 12 are for example arranged on the body or near the body of the operator or in the place of their activities such as the aircraft cockpit. For example, these sensors 12 are arranged in a fixed and/or removable manner in the cockpit of the aircraft piloted by the operator.

Each sensor 12 allows to provide a data type determined as a function of the nature of this sensor 12.

Thus, for example, each sensor 12 allows to provide a data type chosen from the group comprising at least:

Heart rate, for example at different frequencies (60 Hz or 120 Hz for example);

Oxygen saturation;

Electroencephalogram;

Respiratory rate;

Body temperature;

Images;

Acoustic data;

Eye activity (pupillometry, eye opening, gaze direction);

Head position and acceleration;

Blood glucose levels;

Skin conductance (or GSR from the English "Galvanic Skin Response");

Muscle activity (electromyogram, EMG);

Photoplethysmography (PPG);

Functional near-infrared spectroscopy (or fNIRS from the English "Functional near-infrared spectroscopy").

Advantageously, each data type corresponds to a measurable physiological data related to the human body or the environment in which it is located.

Referring to FIG. 1, the determination system 10 comprises an input module 21, a processing module 22, and an output module 23.

The input module 21 allows to acquire all the data generated by the sensors 12. To do this, the input module 21 is connected to each of these sensors by any technically possible means. This means can present direct or indirect connection cables allowing for example the transmission of physiological data acquired by the corresponding sensor via a wired or wireless computer network.

The input module 21 is also connected to a database 25 allowing to store a correspondence matrix and an incidence matrix the meanings of which will be explained in more detail later.

The processing module 22 allows to process all the data acquired by the input module 21 in order to determine the physiological states of the operator.

Furthermore, the processing module 22 allows to determine the correspondence and incidence matrices as will be explained in more detail later.

Finally, the output module 23 allows to provide the processing results of the processing module 22 to any interested system.

Such an interested system comprises for example a human machine interaction interface or any other external system for which the physiological states of the operator are of interest.

Each of the modules 21 to 23 advantageously presents at least partially a software implemented by a processor and stored in a memory.

In such a case, the determination system 10 further comprises such a processor as well as such a memory.

Alternatively, or optionally, each of these modules 21 to 23 presents, at least partially, a programmable logic circuit, for example of the FPGA type (from the English "Field Programmable Gate Array").

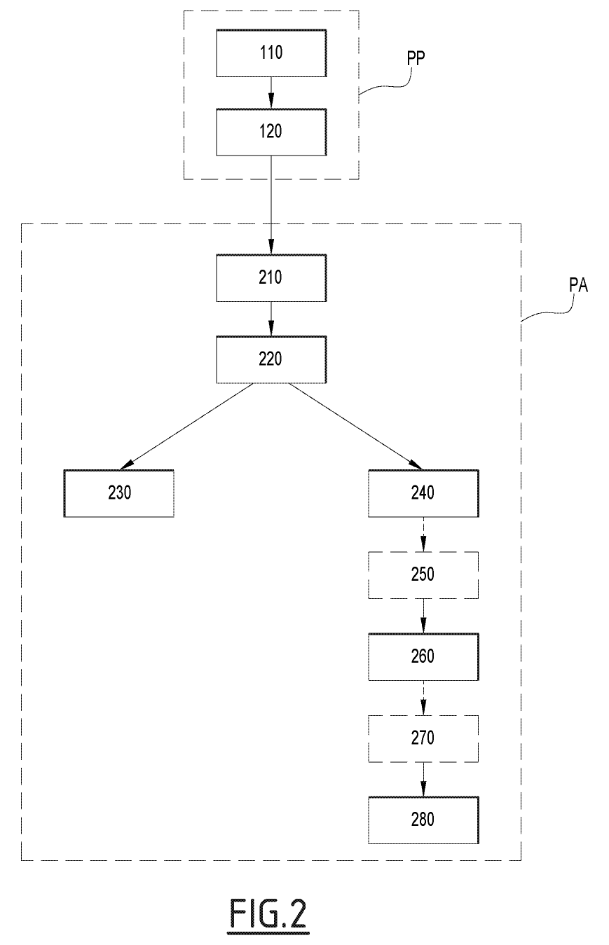

The determination system 10 allows to implement a method for determining the physiological states of the operator which will now be explained with reference to FIG. 2 presenting a flowchart of these steps.

The determination method first comprises a preliminary phase PP during which the correspondence and incidence matrices are determined.

In particular, during a first preliminary step 110, the processing module 22 defines a mathematical model allowing to determine a physiological state of the operator and a set of data types necessary for implementing this mathematical model.

This is done by analyzing each mathematical model used to determine the corresponding physiological state. Each mathematical model is for example known in itself and can be obtained theoretically and/or empirically.

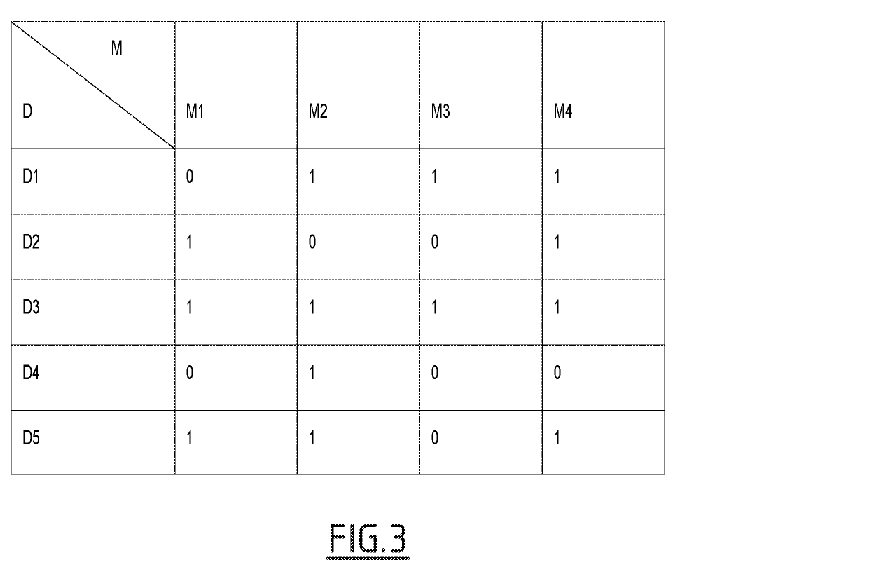

An example of such a determination matrix is illustrated in FIG. 3.

In particular, in the example of this FIG. 3, the correspondence matrix comprises four mathematical models forming the four columns of these matrices allowing to determine the physiological states of the operator.

These models are numbered from M1 to M4 and correspond for example to the determination models of hypoxia, loss of consciousness, stress, and fatigue.

The columns of this correspondence matrix are formed by the data type usable to implement the corresponding mathematical model.

In the example of FIG. 3, five data types numbered from D1 to D5 are represented. These data types correspond for example respectively to heart rate at 60 Hz, heart rate at 120 Hz, oxygen saturation, electroencephalogram, and respiratory rate.

According to the example of FIG. 3, the first model M1 then allows to detect the state of hypoxia from the data D2, D3, and D5 corresponding respectively to: heart rate at 120 Hz, oxygen saturation, and respiratory rate.

According to the same example, the determination model of a loss of consciousness, that is, the second model M2, allows to determine such a loss of consciousness based on data D1, D3, D4, and D5 corresponding respectively to: heart rate at 60 Hz, oxygen saturation, electroencephalogram, and respiratory rate.

In other words, when a data type is necessary to implement the corresponding mathematical model, the correspondence matrix indicates this in the crossing of the corresponding line and column, for example by placing a value "1". Otherwise, a zero value is then placed in such a crossing.

During a second preliminary step 120, the processing module 22 determines an incidence matrix indicating the level of incidence of each physiological state on each other physiological state.

In other words, such an incidence matrix allows to see the correlations between the different states, and notably, allows to determine cases when one physiological state induces another physiological state according to a level of incidence. Such a level of incidence presents, for example, a value between 0 and 1 corresponding to the probability with which a first physiological state induces a second physiological state.

Alternatively, such a value can present the percentage of cases when the first physiological state induces the second physiological state.

When the level of incidence is equal to "0", the first state does not induce the second state. In other words, the second state is independent of the first state.

Conversely, when this level of incidence is equal to "1", the first state always induces the second state. For example, it may be the same state or then physiological states that systematically occur at the same time.

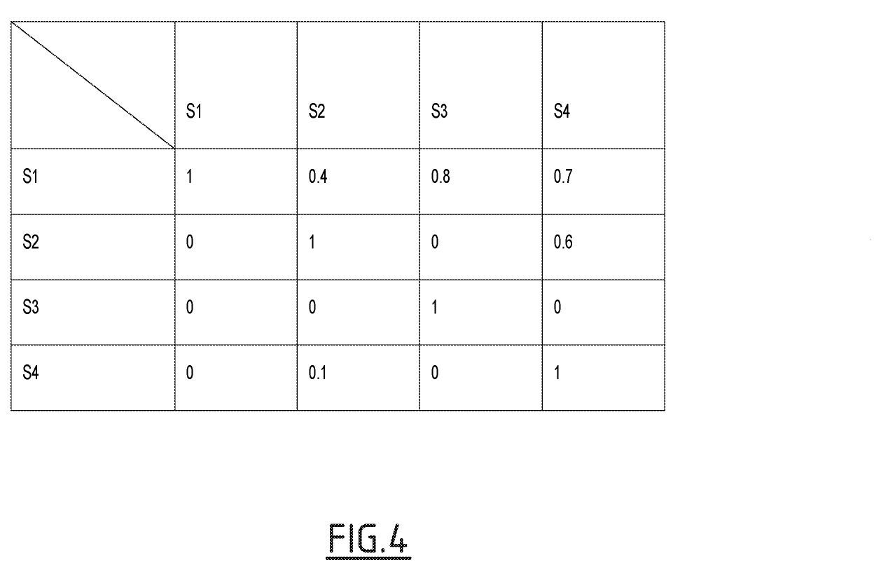

An example of such an incidence matrix is given in FIG. 4.

In the example of this figure, it is then clear that the state of hypoxia, corresponding to the state S1, induces the state of loss of consciousness (state S2) with a probability of 0.4, stress (state S3) with a probability of 0.8, and fatigue (state S4) with a probability of 0.7.

The incidence matrix is, for example, determined by analyzing statistical data as a function of their nature and, for example, as a function of the nature of the mission of the operator. Such an analysis can, for example, comprise the determination of correlations between different physiological states of the operator.

The two preliminary steps 110 and 120 are therefore implemented at least once before the other steps of the determination method.

Alternatively, these two steps are implemented at each update of the determination system 10 when for example a new state and/or a new sensor are integrated into such a system.

The following steps of the method then form an analysis phase PA allowing to determine the physiological states of the operator using the matrices such as defined above. These two matrices are, for example, stored in the database 25 and then accessible at any time by the input module 21.

During an initial step 210 of the analysis phase PA, the input module 21 receives all the measurements provided by the sensors 12.

Then, in some embodiments, the input module 21 implements a preprocessing of these data.

Such preprocessing can comprise data filtering in order to eliminate potential noise, as well as any other type of analog and/or digital preprocessing known, in itself, in the state of the art.

According to some embodiments, during this step, the input module 21 also performs a contextual preprocessing of the data provided by the sensors to determine a number of characteristics related to these data.

These characteristics can comprise for example parameters such as heart rate variations, heart rate peaks, oxygen saturation variations, etc.

This type of contextual processing is also known as such and will not be explained further.

Furthermore, subsequently, by data provided by a sensor, is meant, either raw data provided by the sensor and possibly converted by the input module 21 into digital data, or a characteristic generated by this input module 21 from the raw data provided by the corresponding sensor.

In any case, the nature of the data generated by the input module 21 (in other words, either raw data or characteristics) depends on each mathematical model used by the processing module as will be explained in more detail later.

During a subsequent step 220, the processing module 22 receives all the data generated and/or received by the input module 21 and verifies the availability of each sensor 12.

To do this, the processing module 22 can, for example, analyze the coherence of the data provided by each sensor and when these data are inconsistent, determine that it is then a failing sensor which is then reconsidered as unavailable subsequently.

The processing module 22 can also determine that a sensor is unavailable when no data has been provided by such a sensor.

Alternatively, or in addition, the processing module 22 concludes that a sensor is unavailable using any other information that can be transmitted by external systems.

Thus, for example, a separate sensor operation monitoring system can be used to provide the operating status of each sensor to the processing module 22.

Alternatively, the operator can themselves indicate a failing sensor so that it is then considered by the processing module 22 as an unavailable sensor.

When all the sensors are available, the processing module 22 determines physiological states from each mathematical model using then all the data provided by the input module 21.

Then, during a step 230, the processing module 22 transmits these data to the output module 23 which then provides the determined states to any interested system.

Conversely, when at least one of the sensors is considered unavailable, the processing module 22 implements step 240 during which the processing module 22 excludes from any future consideration each mathematical model using the data type corresponding to such an unavailable sensor.

To do this, the processing module 22 uses the correspondence matrix which indicates for each data type, the mathematical models using this data type to determine the corresponding physiological states.

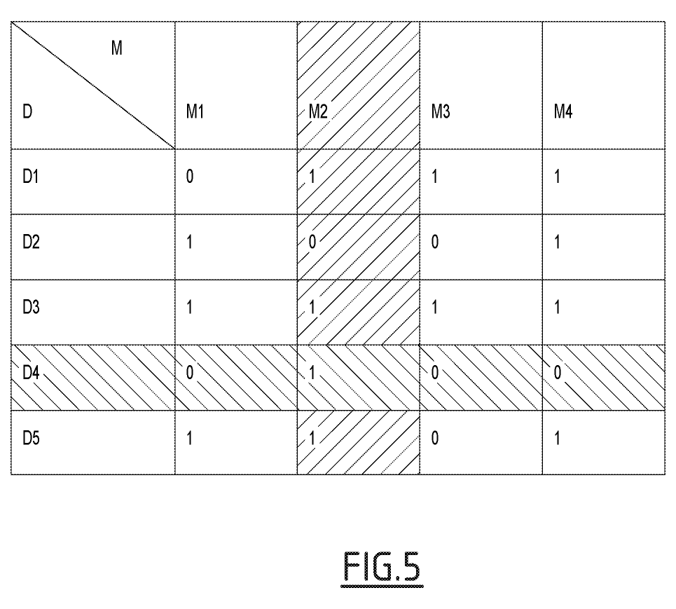

In the example of FIG. 5, the sensor providing the data type D4 is considered unavailable.

Thus, during the step 240, the processing module 22 excludes from consideration the mathematical model M2 corresponding to the only model using the data type D4.

During an optional subsequent step 250, the processing module 22 substitutes the or each mathematical model excluded from consideration during the step 240 with a sub-model allowing to avoid the use of the data type to be provided by the or each unavailable sensor.

The implementation of this step 250 is then conditional as a function of the mathematical model excluded from consideration during the step 240.

In particular, when such an excluded model allows to define a sub-model that does not use the data type to be provided by the unavailable sensor, the step 250 can be implemented in relation to this sub-model.

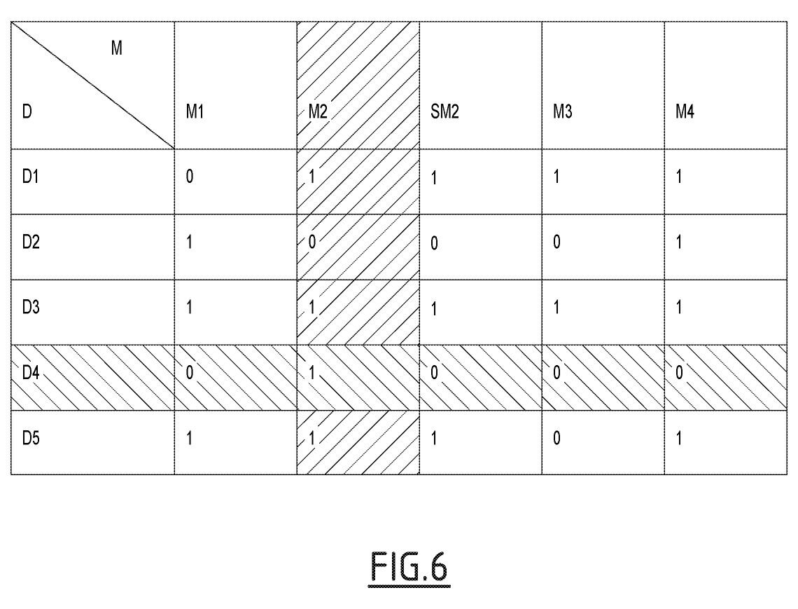

FIG. 6 illustrates such an example in which a sub-model SM2 has been added to the correspondence matrix which can substitute the mathematical model M2 when the data type D4 is unavailable.

During the subsequent step 260, the processing module 22 determines the physiological states of the operator from the mathematical models not excluded from consideration.

In particular, when no mathematical model excluded from consideration during the step 240 has been substituted by a sub-model during the step 250, the processing module 22 uses a reduced number of models to determine the corresponding physiological states.

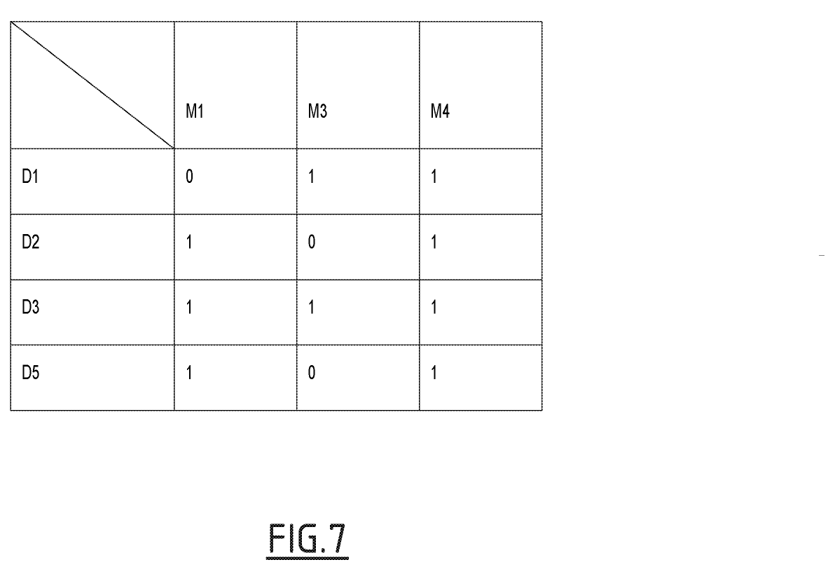

This is illustrated in the example of FIG. 7 on which only the mathematical models M1, M3, and M4 are used following the exclusion of consideration of the model M2.

When, following the exclusion of a mathematical model, the processing module 22 has substituted this excluded model with a sub-model, the processing module 22 then determines during this step 260 the physiological states from all available mathematical models and sub-models determined during the step 250.

Optionally, the physiological state determined during the step 260 by a mathematical sub-model can be correspondingly marked. In other words, marking this physiological state indicating that it was determined from a sub-model and not the usual model can be used.

During the optional subsequent step 270, the processing module 22 determines at least one physiological state corresponding to a mathematical model excluded from consideration during the step 240 and not replaced by a sub-model during the step 250.

To do this, the processing module 22 uses the or each physiological state inducing the physiological state corresponding to the excluded mathematical model, in accordance with the incidence matrix explained previously.

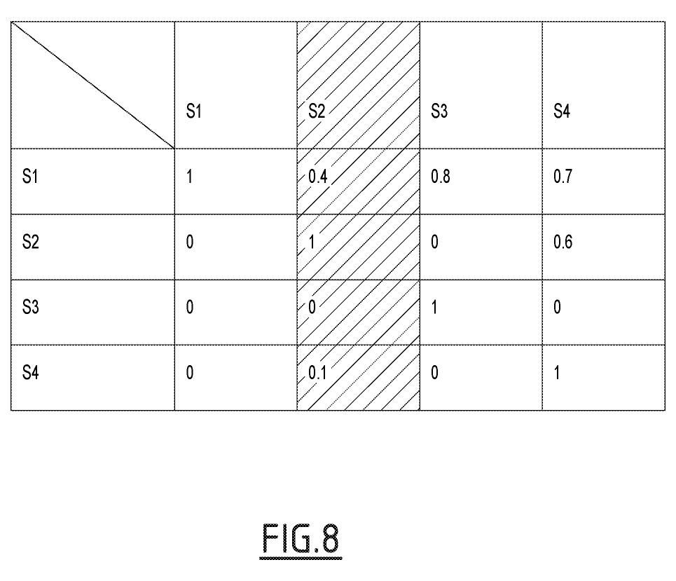

In the example of FIG. 8, the physiological state S2 cannot be determined by the corresponding mathematical model, In other words, by the mathematical model M2 which was then excluded following the loss of the data type D4.

However, in such a case, the processing module 22 can use the physiological state S1 and the physiological state S4 which have respectively the level of incidence on the physiological state S2 equal to 0.4 and 0.1.

To determine the physiological state from the physiological states inducing such a state according to the incidence matrix, the processing module 22 uses, for example, an aggregation model designed for this purpose.

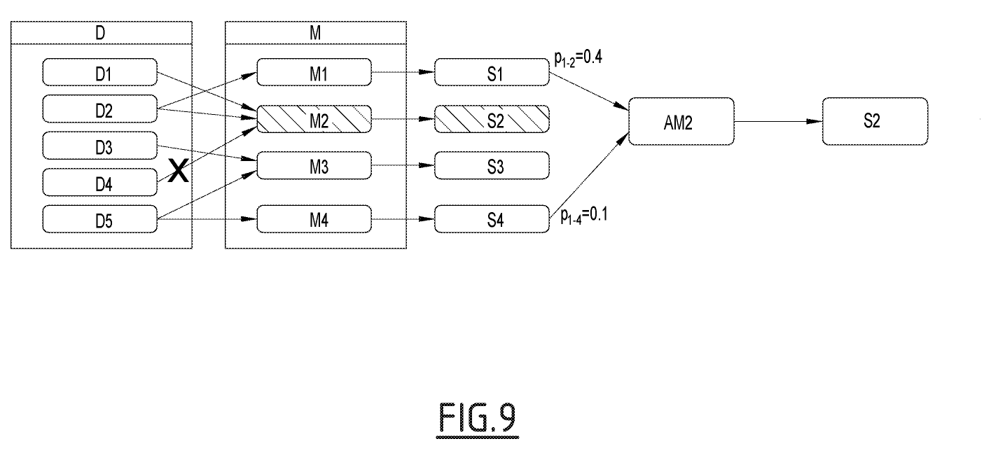

This can then be schematically illustrated in FIG. 9, on which following the loss of the sensor providing the data type D4, the mathematical model M2 cannot be used to determine the physiological state S2. In such a case, the physiological states S4 and S1 are used by an aggregation model AM2 to obtain a physiological state S2.

Advantageously, during this same step 270, the processing module 22 also determines a confidence level of said physiological state determined using other physiological states.

This confidence level is for example determined as a function of the level of incidence of the or each physiological state used by the corresponding aggregation model.

In the example of FIG. 9, the confidence level determined for the physiological state S2 then depends on the incidence levels p₁₋₂ and p₁₋₄ determined in accordance with the incidence matrix.

Finally, during a subsequent step 280, the output module 23 transmits all the determined physiological states to any interested system.

Furthermore, when at least one physiological state has been determined using at least one other physiological state by an aggregation model, the output module 23 also provides the confidence level associated with this physiological state. Similarly, when at least one physiological state has been determined using a sub-model instead of the corresponding model, the output module 23 also provides the corresponding marking.

Then, the steps of the analysis phase PA can be implemented again using other data acquired by the sensors 12.

The present invention then presents a number of advantages.

Firstly, it is clear that, the invention allows to highlight links between the data provided by the different sensors and the physiological states that can be determined from these data. It is also clear that these are different physiological states.

Thus, in the event of the unavailability of at least one of the sensors, the invention allows to exclude or replace easily the mathematical model using the data type derived from this sensor to determine the corresponding physiological state.

This then presents great flexibility of the invention compared to known solutions in the state of the art.

Furthermore, the invention allows to establish links between the different states and in the event of the inability to calculate one of the states by the corresponding mathematical model to use the other states to then estimate this last state.

In such a case, a confidence level can be provided to warn any other interested system that the corresponding physiological state has been obtained by indirect means.

1. A method for determining physiological states of the operator;
the method comprising a first preliminary step of determining a correspondence matrix between a mathematical model allowing to determine a physiological state of the operator and a set of data types necessary for implementing the mathematical model;
the method further comprises the following steps:
verification of the availability of each sensor allowing to provide a data type;
when a sensor is unavailable, exclusion of consideration of each mathematical model using the data type corresponding to this sensor, in accordance with the correspondence matrix;
determination of the physiological states of the operator from the mathematical models not excluded from consideration.

2. The method according to claim 1, further comprising a step of substituting at least one mathematical model excluded from consideration with a mathematical sub-model allowing to determine the same physiological state from a set of data types without the data type of the unavailable corresponding sensor.

3. The method according to claim 1, further comprising a second preliminary step of determining an incidence matrix indicating the level of incidence of each physiological state on each other physiological state.

4. The method according to claim 3, wherein each level of incidence corresponds to the probability of transitioning from one physiological state to another physiological state.

5. The method according to claim 3, further comprising a step of determining at least one physiological state corresponding to a mathematical model excluded from consideration, by an aggregation model using the or each physiological state inducing this physiological state corresponding to the mathematical model excluded from consideration, in accordance with the incidence matrix.

6. The method according to claim 5, wherein said physiological state corresponding to the mathematical model excluded from consideration is determined with a confidence level determined as a function of the level of incidence of the or each physiological state used by the aggregation model.

7. The method according to claim 1, wherein each physiological state is chosen from the group comprising at least:
hypoxia;
loss of consciousness;
stress;
fatigue;
spatial disorientation;
blackout;
dehydration;
mental load;
mind wandering;
hyperventilation;
hypoglycemia;
visual or attentional tunneling.

8. The method according to claim 1, wherein each data type is chosen from the group comprising at least:
Heart rate, notably at different frequencies;
Oxygen saturation;
Electroencephalogram;
Respiratory rate;
Body temperature;
Images;
Acoustic data;
Eye activity;
Head position and acceleration;
Blood glucose levels;
Skin conductance;
Muscle activity;
Photoplethysmography;
Functional near-infrared spectroscopy.

9. A computer program including software instructions which, when executed by a computer, implement a method according to claim 1.

10. A system for determining the physiological states of the operator comprising an input module, a processing module and an output module configured to implement a method according to claim 1.
